(19) United States
(12) Patent Application Publication
(10) Pub. No.: US 2004/0259087 A1
(43) Pub. Date: Dec. 23, 2004

(54) Use

(76) Inventors: David Campbell, Hertfordshire (GB); Nigel Spurr, Essex (GB); Ralph McGinnis, Essex (GB); Ana Maria Valdes, Essex (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/415,682

(22) PCT Filed: Nov. 2, 2001
(86) PCT No.: PCT/GB01/04876

(30) Foreign Application Priority Data
Nov. 4, 2000 (GB) 0027181.7

(51) Int. Cl. C12Q 1/68; C07H 21/04; C12P 19/34

(52) U.S. Cl. 435/6; 435/91.2; 536/24.3

(57) ABSTRACT

The invention relates to diagnostic methods and kits which are suitable for determining the presence, in a subject, of Lp-PLA₂ polymorphic variants which are associated with a higher incidence of atherosclerosis, and to the use of such methods and kits.

The present invention relates to polymorphic variants of the lipoprotein-associated phospholipase A₂ (Lp-PLA₂), which are associated with a higher incidence of atherosclerosis in those patients that carry the variant forms. The invention concerns diagnostic methods and kits which are suitable for determining the presence of said Lp-PLA₂ polymorphic variants in a patient, and to the use of such methods and kits.

Prevention of disease or early therapy of disease is a highly desirable goal of health management organizations, health care providers, and of course the patients themselves. Early therapeutic intervention can lead to prevention, improved outcome and/or reduced length and cost of treatment. Such early intervention necessitates the accurate identification of patients at risk from a particular disease. Traditionally patients at risk have been identified according to phenotypic parameters (frequently after some phenotypic abnormality has manifested itself) or in some cases genetically if they are members of a family with a history of the disease condition. However recently it has become possible to accurately identify patients at risk at a much earlier stage and without the need to depend on phenotypic cues or family history. All that is required is a sample of the patient's blood, for example, which can then be genotyped to determine whether the individual carries a particular genetic polymorphism know to be associated with a particular disease state.

Such genotyping obviously requires knowledge of an association between a genetic polymorphism and a disease state. The determination of such associations is presently an active area of research.

As herein used the term genetic polymorphism is defined as a naturally occurring variation in the DNA sequence of an organism which may or may not result in phenotype variation. ‘Phenotype’ may be defined as the combined physical characteristics of the organism and includes (but not exclusively) disease states in humans. Polymorphisms can be divided into two broad classes: single base substitutions (also known as single nucleotide polymorphisms or SNPs), and deletion/insertion events.

An SNP occurs when a specific nucleotide position within the DNA sequence has two or more states in the population under study, ie a different nucleotide may be present at the given position when different individuals are compared. SNPs can occur within genes (where ‘gene’ is defined as the entire coding and regulatory regions giving rise to a specific protein). Such intragenic polymorphisms may or may not directly affect gene or protein function. SNPs may also lie outside genes (extragenic).

Deletion/insertion polymorphisms occur when one or more nucleotides is absent in one individual when compared to another. The most common type of insertion/deletion polymorphism used in genetic analysis is the tandem repeat sequence, where a specific stretch of DNA within the genome consists of a tandemly repeated motif. Such sequences show variability (polymorphism) in the number of repeats, resulting in different lengths of DNA fragment in different individuals. Tandem repeat polymorphisms can be divided into two categories, depending on the number of nucleotides comprising the repeat unit (n). The two classes are variable number of tandem repeat loci (VNTRs) where n>4 and simple tandem repeat loci (STRs) where n<5. Both VNTRs and STRs can be used for genetic association studies. Tandem repeats typically lie outside genes but can also occur within genes.

Both intra- and extragenic polymorphisms can be used for the identification of genetic associations with phenotype. An intragenic polymorphism may have a direct influence on phenotype by altering the level of gene expression or the structure of the resultant protein. Alternatively, an intragenic or extragenic polymorphism of no direct functional consequence may be physically linked to a second polymorphism which is of functional significance, allowing a test for association with a phenotype indirectly, in the absence of any knowledge of the functional variant itself.

In addition to the use of genetic polymorphisms for the identification of patients at risk, such genotypic knowledge can be used to select patients groups for clinical trial studies, and also to interpret the results of such trials. Essentially the statistical power of clinical trial studies to detect efficacy of a therapeutic agents can be improved if appropriate knowledge of prognostic factors that can influence response to therapy is included as part of the study design.

In so far that genetic polymorphisms can be used as prognostic factors in clinical studies, the knowledge and assay of such polymorphisms has the potential of making some of these studies more cost effective. This is true if the inclusion of genetic prognostic factors translates in equivalent statistical power to detect efficacy with the smaller number of patients, thus decreasing the cost of a given study.

Lp-PLA₂ is a secreted, calcium-independent member of the growing phospholipase A2 superfamily (Tew et al (1996) Arterioscler Thromb Vasc Biol. 16(4):591-9; Tjoelker et al (1995) Nature 374(6522):549-53). It is produced by monocytes, macrophages, and lymphocytes and is found associated predominantly with LDL (−80%) in human plasma. The enzyme cleaves polar phospholipids, including PAF, and is also known as PAF acetylhydrolase (Tjoelker et al (1995) supra).

Many observations have demonstrated a pro-inflammatory activity of oxidised LDL when compared with native unmodified lipoproteins. One of the earliest events in LDL oxidation is the hydrolysis of oxidatively modified phosphatidylcholine, generating substantial quantities of lysophosphatidylcholine (lyso-PC) and oxidised fatty acids. This hydrolysis is mediated solely by Lp-PLA₂. A significant amount of evidence has accumulated in favour of lyso-PC being a proinflammatory and proatherogenic mediator. In addition to being cytotoxic at higher concentrations it is able to stimulate monocyte and T-lymphocyte chemotaxis, as well as induce adhesion molecule and inflammatory cytokine expression at more modest concentrations. Lyso-PC has also been identified as the component of oxidised LDL that is involved in the antigenicity of LDL, a feature that may also contribute to the inflammatory nature of atherosclerosis. Moreover, lyso-PC promotes macrophage proliferation and induces endothelial dysfunction in various arterial beds. The oxidised fatty acids that are liberated together with lyso-PC, are also monocyte chemoattractants and may also possess many more relevant biological activities (e.g. cell signalling). Thus inhibition of Lp-PLA₂ should retard atherosclerosis by interfering with inflammatory cell localization, activation, pro-inflammatory function and death.

Lp-PLA₂ has been found to be enriched in the highly atherogenic lipoprotein subfraction of small dense LDL, which is very susceptible to oxidative modification. Moreover, enzyme levels are increased in patients with hyperlipidaemia, stroke, Type 1 and Type 2 diabetes mellitus, as well as in post-menopausal women. As such, plasma Lp-PLA₂ levels tend to be elevated in those individuals who are considered to be at risk of developing accelerated atherosclerosis and clinical cardiovascular events.

The current primary therapy for atherosclerotic disease is aggressive plasma cholesterol lowering and is dominated by use of the HMG-CoA reductase inhibitors, the statins. Overall, 50% of patients with cardiovascular disease are hypercholesterolaemic. However in many cases the effectiveness of such treatments may be severely compromised by, for example, late diagnosis of the condition. Thus a patient may already have advanced atherosclerotic plaques before treatment commences, reducing the prospect of a successful therapeutic outcome. In addition, early diagnosis can enable preventative measures to be put in place, for example changes in aspects of lifestyle and diet that are known non-genetic risk factors for the development of atherosclerosis. Therefore it is clear that there is a need for the development of new and effective diagnostic methods and indeed for new and effective treatments for atherosclerosis and hypercholesterolaemia. However these new and effective treatments are dependent on the identification of new and useful gene and/or protein targets which have a demonstrated link to the disease. These targets can be isolated and screens developed in order to identify compounds useful in the treatment of atherosclerosis.

The present invention is based upon the finding that certain polymorphic variants of the Lp-PLA₂ gene are associated with an increased incidence of atherosclerosis in humans.

In a first aspect the invention provides a method for diagnosing atherosclerosis in a subject, or for predicting the susceptibility of a subject to atherosclerosis, comprising determining the presence or absence of a single nucleotide polymorphism (SNP) in codon 379 of a Lp-PLA₂-encoding polynucleotide isolated from the subject, wherein the codon comprising the SNP encodes an amino acid other than valine.

In a preferred embodiment codon 379, comprising the SNP, encodes the amino acid alanine.

In a more preferred embodiment the SNP is a cytosine residue located at the second nucleotide position of the triplet of nucleotides making up codon 379, that is at a position corresponding to nucleotide residue 1173 of the Lp-PLA₂ cDNA sequence of SEQ ID NO: 1. At a “position corresponding to”, in the context of the present invention, is defined as the second nucleotide position in the codon encoding the amino acid residue shown at position 379 in the polypeptide sequence of SEQ ID NO:2.

The Lp-PLA₂ cDNA of SEQ ID NO:1 was derived from a lymphoma library (Tew et al (1996) supra) and has a cytosine residue (C) at nucleotide position 1173 which results in a GCA codon which encodes alanine as shown in the polypeptide sequence of SEQ ID NO:2. An alternative variant is known to have a thymine (T) residue at this position which results in a GTA (valine encoding) codon (Tjoelker et al (1995) supra).

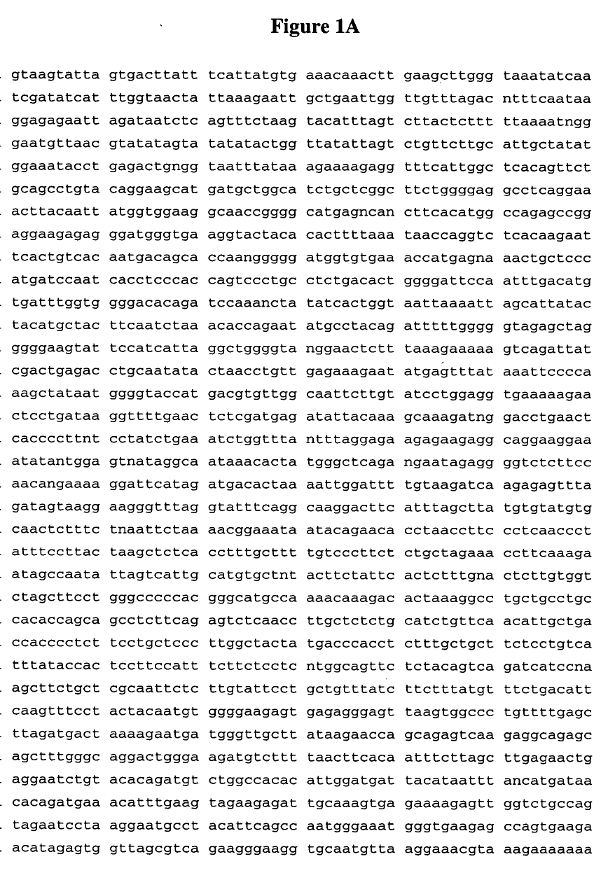

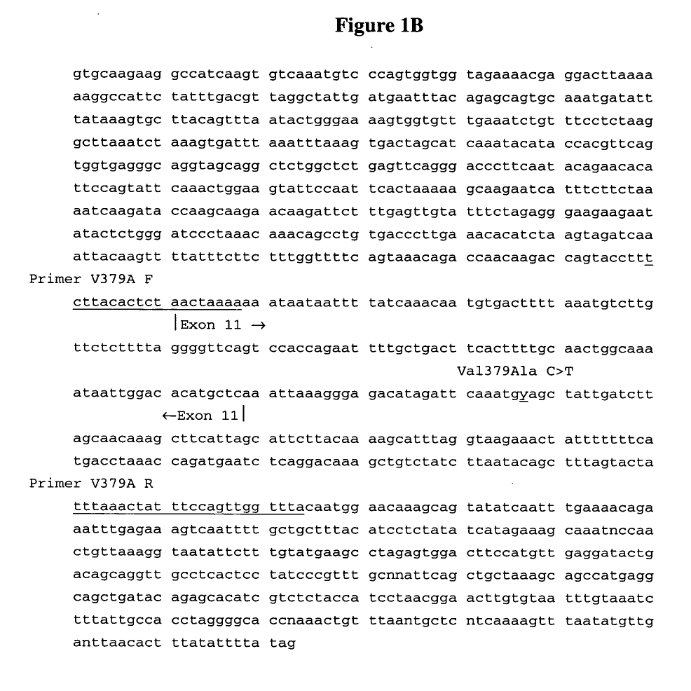

The polynucleotide of SEQ ID NO:3 (annotated in FIG. 1.) is a portion of the genomic sequence of Lp-PLA₂ which shows exon 11 (nt 2711 to 2860), wherein lies the codon for amino acid 379, together with flanking intronic sequence. In this sequence the polymorphic base is at position 2807 and is marked as “y” (ie. pyrimidine; C or T). It will be clear to the skilled person that the variant nucleotide at position 1173 of the cDNA sequence of SEQ ID NO:1 is the same nucleotide as the variant nucleotide at position 2807 in the genomic DNA sequence of SEQ ID NO:3.

Other polymorphic variants of Lp-PLA₂ are known wherein the nucleotide differences are at positions other than in codon 379, for example codon 279 (WO95/09921, ICOS Corporation) wherein the variants have phenylalanine or valine at this position. Phenylalanine at position 279 was found to severely affect the activity of the enzyme and is believed to be associated with severe respiratory symptoms in asthmatic children.

The polymorphisms of the present invention are considered independently of any other Lp-PLA₂ polymorphism. Thus the diagnostic method of the invention is designed to detect only the polymorphism at codon 379, whether or not further Lp-PLA₂ polymorphisms are present in the subject's Lp-PLA₂ gene.

Preferably the diagnostic method of the invention involves the use of a DNA amplification method, preferably a polymerase chain reaction (PCR)-based DNA amplification method.

In one embodiment PCR primers are provided which are specific to the polymorphic variant to be detected. The design of such primers for diagnostic purposes as described herein is well known in the art. Thus, for example, one of the two primers may have at the 3′ end a complementary base to the variant such that DNA amplification between the two primers is achieved only when the variant nucleotide is present in the DNA of the patient. A control primer having the alternative (typically the “normal”) nucleotide at this position is included as a positive control. It is of course well known in the art that humans have two copies of each gene sequence (except for males who have only a single copy of the genes on the X and Y chromosomes) and thus in a particular human subject the polymorphic variant may be present in neither, one or both copies of the Lp-PLA₂ gene carried by that subject. A subject carrying identical copies of the gene are described as being homozygous for that gene. Where the two copies are different, for example one copy carries the Lp-PLA₂-A379 polymorphism whereas the other copy carries the Lp-PLA₂-V379 form, the subject is heterozygous. When the DNA amplification products are separated on a gel or by chromatography, for example HPLC, the DNA band pattern using primers designed as described above will differ according to whether the subject being tested is homozygous for either polymorphic variant or is heterozygous. The interpretation of the DNA band patterns is well known to the skilled person.

In a preferred embodiment the diagnostic method involves the use of PCR primers designed as follows:

1) the 3′ primer” is between 10 and 30 nucleotides in length, preferably 15 to 25, most preferably 20, and is fully complementary to the DNA sequence of the Lp-PLA₂ sequence up to and including the C nucleotide at position 1173 of SEQ ID NO:1 (thus the most 3′ complementary nucleotide in the primer will be a G);

2) the “5′ primer” is of similar length to the 3′ primer, between 10 and 30 nucleotides in length, preferably 15 to 25 nucleotides, most preferably 20 nucleotides and which is a direct copy of the relevant part of the Lp-PLA₂ sequence of SEQ ID NO:1 suitably positioned 5′ to the first primer such that under amplification conditions, the DNA between the two primers is amplified when the C1173 polymorphism is present in at least one copy in the individual's genome;

3) in such tests it is usual to provide a second 3′ primer, to be used in a second, independent, reaction together with the common 5′ primer of (2) which is designed to have as it's most 3′ complementary nucleotide the alternative polymorphic nucleotide (in this case A, this being the complementary nucleotide to T1173 of SEQ ID NO:1).

The results of a diagnostic test using these 3 primers would be:

a) a C allele (ala379) homozygote will give a PCR band using 3′ primer (1) but no band with the 3′ primer (3);

b) a T allele (val379) homozygote will give a PCR band using 3′ primer (3) but no band with the 3′ primer (1);

c) a C/T (ala/val379) heterozygote will give bands with both 3′ primer (1) and (3) reactions.

In the instance described above the primer having the polymorphic nucleotide 1173 at the 3′ end is the 3′ primer. It will be appreciated by the skilled man that the primer having the polymorphic nucleotide 1173 at the 3′ end could instead be the 5′ primer.

An alternative diagnostic method involves using primer pairs which flank, but do not overlap with the polymorphic nucleotide. In this test the primers amplify a fragment of the Lp-PLA₂ sequence which includes the polymorphic nucleotide and thereafter the differences in electrophoretic mobility of the amplified DNA are detected. For example, the DNA fragment carrying the C1173 allele will migrate differently to that carrying the T1173 allele. Detection of the polymorphic variant forms may be carried out using, for example, dHPLC (O'Donovan, M C et al (1998) Genomics, 52(1):44-49; Underhill, P A et al (1997) Genome Res. 7(10):996-10005). In a preferred embodiment the primers have the sequences shown in FIG. 1 as V379A F (the forward primer consisting of nucleotides 2640 to 2658) and V379A R (the reverse primer consisting of nucleotides 2964 to 2941). FIG. 1 shows the exon 11 sequence (nucleotides 2711 to 2860) and flanking intronic sequence with the variant base (2807) marked as “y” and the aforementioned primer sequences underlined.

In order to carry out the method of the invention a sample comprising a Lp-PLA₂ polynucleotide must be isolated from the subject. Nucleic acids for diagnosis may be obtained from a subjects cells, such as from blood, urine, saliva, tissue biopsy or autopsy material. The genomic DNA may be used directly for detection or it may be amplified enzymatically by using PCR, preferably RT-PCR, or other amplification techniques prior to analysis. RNA or cDNA may also be used in similar fashion. The polymorphic variants can be identified by hybridizing amplified DNA to labeled Lp-PLA₂-A379 or Lp-PLA₂-V379 nucleotide sequences. Perfectly matched sequences can be distinguished from mismatched duplexes by RNase digestion or by differences in melting temperatures. DNA sequence difference may also be detected by alterations in the electrophoretic mobility of DNA fragments in gels, with or without denaturing agents, or by direct DNA sequencing (see, for instance, Myers et al., Science (1985)230:1242). Sequence changes at specific locations may also be revealed by nuclease protection assays, such as RNase and S1 protection or the chemical cleavage method (see Cotton et al., Proc Natl Acad Sci USA (1985) 85: 4397-4401).

In a further aspect, the present invention relates to a diagonostic kit for performing the diagnostic method of the invention comprising:

(a) a polynucleotide of the present invention, preferably the nucleotide sequence of SEQ ID NO:1, SEQ ID NO:3 a fragment or an RNA transcript thereof; and

(b) a nucleotide sequence complementary to that of (a).

Preferably the polynucleotides (a) and (b) of the diagnostic kit are oligonucleotide primers for use in PCR reactions as described hereinabove.

Most preferably the diagnostic kit comprises two or more primers wherein at least one primer has the polymorphic nucleotide 1173 as the 3′ nucleotide. Still more preferably the kit comprises 3 primers wherein two primers are identical but for the 3′ nucleotide, one of these primers corresponding to the C1173 allele (giving ala379) and the other to the T1173 allele (giving val379).

In an alternative embodiment the diagnostic kit comprises two PCR primers that flank the polymorphic nucleotide at 1173 (2083 in SEQ ID NO:3/FIG. 1). Preferably the PCR primers have the sequences shown in FIG. 1 as V379A F (the forward primer consisting of nucleotides 2640 to 2658) and V379A R (the reverse primer consisting of nucleotides 2964 to 2941).

The diagnostic methods and diagnostic kits of the invention can be used for

a) predicting the likelihood of developing atherosclerosis;

b) predicting and responding to the progression of the atherosclerotic condition;

c) predicting and responding to reaction to drug treatment; or

d) predicting disease outcome.

in a subject.

In a further aspect the diagnostic methods and diagnostic kits of the invention may also be used for the selection of patient groups for conducting clinical trials concerning therapeutic compounds with potential for use in the treatment of atherosclerosis.

The invention will now be illustrated by the following examples.

### EXAMPLES

### Example 1

Human Subjects and Study Design

Caucasian individuals from the USA were tested for coronary artery calcification (CAC) using EBCT, plasma cLDL, HDL and Lp-PLA2. Subjects were selected to have a family history of coronary heart disease (at least on first degree relative with premature CHD) and absence of common risk factors. The technique to measure atherosclerosis, electron beam computed tomography (EBCT), is a sensitive and specific measure of coronary artery calcification, and is valuable for non-invasive quantification of atherosclerotic plaque size (Circulation 93:1951-53; 1996; Mayo Clin. Proc 71:369-77;1996).

### Example 2

Genotyping

Sequencing-by-synthesis using Pyrosequencing™ technology (Ahmadian, A et al (2000) Anal. Biochem. 280(1):103-110; Nordstrom, T et al (2000) Biotechnol. Appl. Biochem. 31(2):107-112) was performed using primers to produce a biotinylated PCR product flanking the Val379Ala variant in exon 11 of the Lp-PLA2 gene. PCR was conducted using the following conditions: 95° C. for 10 minutes, 50 cycles each of 95° C. for 30 seconds, 55° C. for 60 seconds and 72° C. for 60 seconds, followed by a final cycle of 72° C. for 10 minutes in 20 μl reactions containing 10 pmoles of each primer, 1 U AmpliTaq Gold (Perkin Elmer), 0.2 mM dNTPs (Promega), 15 mM Tris.HCl pH 8.0, 50 mM KCl, 2.5 mM MgCl₂ solution, plus 50 ng of DNA. Biotinylated PCR products were immobilized to streptavidin-coated Dynabeads™ (M280-Streptavidin, Dynal) by incubating 125 μg Dyanbeads with 5 pmoles of PCR product by agitation for 30 minutes at 43° C. The Dynabeads were transferred to a LucPlate™ 96 and after washing, the strands separated by denaturing the DNA in 0.3M NaOH for 5 minutes. The immobilized strand was washed and annealed with 15 pmoles of sequencing primer in 40 μl annealing buffer at 95° C. for 1 minute followed by cooling at room temperature. LucKit™ SNP 96 reagents were placed in the Luc 96 cassette according to Pyrosequencing protocols. The cassette and the Luc96 plate containing the magnetic beads with the primer annealed to immobilized single stranded DNA were placed in the Luc96 instrument for sequencing-by-synthesis. The nucleotide sequence was determined from the signal peaks in the pyrogram produced by the Luc96 instrument. The oligonucleotides used to investigate Val379Ala polymorphism in the Lp-PLA2 gene were:

V379A F 5′ TCCTTACACTCTAACTAAAA,

5′ biotin labelled reverse oligo V379A R 5′ TAAACCAACTGGAAATAGTT, and

sequencing primer V379A 5′ GGAGACATAGATTCAAATG.

### Example 3

Measurement of LDL and HDL Levels in Plasma

LDL and HDL levels in plasma were performed by a modification of the standard Lipid Research Clinic's protocol (US Department Health, Education and Welfare. Lipid Research Clinics Program: Manual of Laboratory Operations Vol. 1: Lipid and Lipoprotein Analysis (publication no. (NIH) 75-628). Bethesda, Md.: National Institutes of Health, 1975).

Results

TABLE 1
LDL, HDL and LDL/HDL ratio by
LpPLA2 V379A genotype (women):

| | LpPLA2 | LDL | HDL | LDL/ | | |
|---|---|---|---|---|---|---|
| Women | genotype | mg/dl | mg/dl | HDL | sd | n |
| No statins | Val Val | 125.11 | 60.84 | 2.21 | 0.81 | 64 |
| | Val Ala | 128.68 | 64.52 | 2.19 | 1.18 | 41 |
| | Ala Ala | 144.68 | 46.40 | 3.21 | 1.11 | 5 |
| Statins | Val Val | 112.44 | 56.67 | 1.97 | 0.63 | 11 |
| | Val Ala | 109.93 | 54.67 | 2.07 | 0.80 | 3 |
| | Ala Ala | — | — | — | — | 0 |

TABLE 2
LDL, HDL and LDL/HDL ratio by
LpPLA2 V379A genotype (men):

| | LpPLA2 | LDL | HDL | LDL/ | | |
|---|---|---|---|---|---|---|
| Men | genotype | mg/dl | mg/dl | HDL | sd | n |
| No statins | Val Val | 130.22 | 42.79 | 3.19 | 0.93 | 70 |
| | Val Ala | 136.11 | 45.49 | 3.06 | 0.83 | 54 |
| | Ala Ala | 136.00 | 35.71 | 3.96 | 1.40 | 7 |
| Statins | Val Val | 105.63 | 41.08 | 2.61 | 0.84 | 24 |
| | Val Ala | 114.98 | 39.67 | 3.00 | 1.08 | 12 |
| | Ala Ala | 138.73 | 39.67 | 3.44 | 0.92 | 3 |

TABLE 3
LpPLA2 plasma levels by V379A genotype

| Genotype | | | |
|---|---|---|---|
| (site 379) | Gender | Plasma LpPLA2 ug/ml | s.e. |
| Val/Val | Women | 2.06 | 0.07 |
| Val/Ala | Women | 2.06 | 0.09 |
| Ala/Ala | Women | 2.39 | 0.24 |
| Val/Val | Men | 2.10 | 0.06 |
| Val/Ala | Men | 2.17 | 0.07 |
| Ala/Ala | Men | 2.46 | 0.17 |

TABLE 4
Levels of CAC measured by EBCT by
Lp PLA2 genotype (V379A)

| Gender | statins | n | Genotype | log EBCT | std dev | std err |
|---|---|---|---|---|---|---|
| Women | No | 6 | Ala/Ala | 1.71 | 2.49 | 1.02 |
| Men | No | 6 | Ala/Ala | 3.47 | 2.93 | 1.20 |
| Women | No | 121 | Val+ | 0.78 | 1.86 | 0.17 |
| Men | No | 153 | Val+ | 2.20 | 2.31 | 0.19 |

The Val379Ala polymorphism was found to show the following associations with clinical endpoints:

Homozygosity for alanine at position 379 was more common among the subjects with CAC (8.2%) than those without CAC (3.6%). Ala/Ala homozygotes were found to have:

15% higher levels of Lp PLA2 than the Valine carriers (Ala/Val and Val/Val (p<0.02))

26% (men) to 45% (women) higher LDL/HDL ratios than Valine carriers (p<0.01).

Higher levels of CAC than Valine carriers (not statistically significant but consistent with the other results).

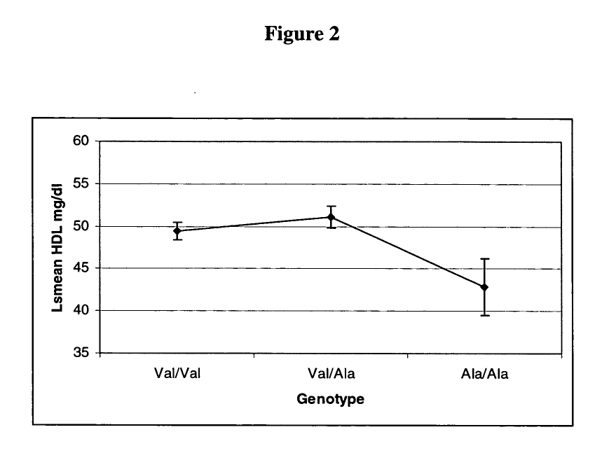

In a further, separate, experiment significant effects in the model were gender, smoking status and BMI were noted. There was a significant relationship between genotype and HDL level (p<0.05). Two individual genotype comparisons were also significant (Val/Val versus Ala/Ala, p<0.05 and Val/Ala versus Ala/Ala, p<0.02). In both cases the mean HDL (adjusting for other factors in the model) of the Ala/Ala homozygotes was lower than that of the other group (see FIG. 2). There was no statistically significant difference between the mean HDL of Val/Val versus that of Val/Ala.

There was a statistically significant association between Valine carrier status and HDL levels (p<0.04). This corresponds to a mean HDL (adjusting for other factors in the model) for the Valine carriers of 42.9 mg/dl and a mean of 50.0 mg/dl for the Ala/Ala homozygotes. A similar analysis was performed using Logistic Regression. The same factors were included in the analysis model. The addition of genotype was significant (p<0.03). The addition of allele was also significant (p<0.05).

To investigate this result further, t-tests were performed to compare the mean HDL of the different genotypes. There was a significant difference in the mean HDL of Val/Val versus Ala/Ala (p<0.003) and of Val/Ala versus Ala/Ala (p<0.005). There was no difference between the means of the Val/Val and Val/Ala groups. When the Val/Val and Val/Ala data are combined there is a significant difference between the mean HDL of this group and the mean of the Ala/Ala group (p<0.002). It is important to note that the t-test does not adjust for any other factor.

1-11 cancel

12. A method for diagnosing atherosclerosis in a subject, or for predicting the susceptibility of a subject to atherosclerosis, comprising determining the presence or absence of a single nucleotide polymorphism (SNP) in codon 379 of a lipoprotein-associated phospholipase A2 (Lp-PLA₂)-encoding polynucleotide isolated from the subject, wherein the codon comprising the SNP encodes an amino acid other than valine.

13. A method according to claim 12 wherein codon 379, comprising the SNP, encodes the amino acid alanine.

14. A method according to claim 13 wherein the SNP is a cytosine residue located at the second nucleotide position of the triplet of nucleotides making up codon 379, that is at a position corresponding to nucleotide residue 1173 of the Lp-PLA₂ cDNA sequence of SEQ ID NO:1.

15. A method according to claim 12 comprising a DNA amplification method.

16. A diagnostic kit for carrying out the method of claim 12.

17. A diagnostic kit according to claim 16 comprising:
1) a 3′ primer” complementary to the DNA sequence of the Lp-PLA₂ sequence up to and including the C nucleotide at position 1173 of SEQ ID NO:1; and
2) a “5′ primer” which is a direct copy of part of the Lp-PLA₂ sequence of SEQ ID NO:1 suitably positioned 5′ to the 3′ primer such that under amplification conditions, the DNA between the two primers is amplified when the C1173 polymorphism is present in at least one copy in the individual's genome.

18. A diagnostic kit according to claim 17 further comprising an additional 3′ primer complementary to the DNA sequence of the Lp-PLA₂ sequence up to and including the T nucleotide at position 1173 of SEQ ID NO:1.

19. A diagnostic kit according to claim 16 comprising primers flanking the polymorphic nucleotide at position 1173 of SEQ ID NO:1.

20. A diagnostic kit according to claim 19 wherein the primers are V379A F and V379A R.

21. Use of the method of claim 12 for:
a) predicting the likelihood of developing atherosclerosis;
b) predicting and responding to the progression of the atherosclerotic condition;
c) predicting and responding to reaction to drug treatment; or
d) predicting disease outcome.
in a subject.

22. Use of a method of claims 12 for the selection of patient groups for conducting clinical trials concerning therapeutic compounds with potential for use in the treatment of atherosclerosis.


SEQUENCE LISTING (6 sequences)

SEQ ID NO: 1 - DNA, 1323 bp, Homo sapien
atggtgccacccaaattgcatgtgcttttctgcctctgcggctgcctggctgtggtttatccttttgactggcaatacataaatcctgttgcccatatgaaatcatcagcatgggtcaacaaaatacaagtactgatggctgctgcaagctttggccaaactaaaatcccccggggaaatgggccttattccgttggttgtacagacttaatgtttgatcacactaataagggcaccttcttgcgtttatattatccatcccaagataatgatcgccttgacaccctttggatcccaaataaagaatatttttggggtcttagcaaatttcttggaacacactggcttatgggcaacattttgaggttactctttggttcaatgacaactcctgcaaactggaattcccctctgaggcctggtgaaaaatatccacttgttgttttttctcatggtcttggggcattcaggacactttattctgctattggcattgacctggcatctcatgggtttatagttgctgctgtagaacacagagatagatctgcatctgcaacttactatttcaaggaccaatctgctgcagaaataggggacaagtcttggctctaccttagaaccctgaaacaagaggaggagacacatatacgaaatgagcaggtacggcaaagagcaaaagaatgttcccaagctctcagtctgattcttgacattgatcatggaaagccagtgaagaatgcattagatttaaagtttgatatggaacaactgaaggactctattgatagggaaaaaatagcagtaattggacattcttttggtggagcaacggttattcagactcttagtgaagatcagagattcagatgtggtattgccctggatgcatggatgtttccactgggtgatgaagtatattccagaattcctcagcccctcttttttatcaactctgaatatttccaatatcctgctaatatcataaaaatgaaaaaatgctactcacctgataaagaaagaaagatgattacaatcaggggttcagtccaccagaattttgctgacttcacttttgcaactggcaaaataattggacacatgctcaaattaaagggagacatagattcaaatgcagctattgatcttagcaacaaagcttcattagcattcttacaaaagcatttaggacttcataaagattttgatcagtgggactgcttgattgaaggagatgatgagaatcttattccagggaccaacattaacacaaccaatcaacacatcatgttacagaactcttcaggaatagagaaatacaat

SEQ ID NO: 2 - protein, 441 aa, Homo sapien
MVPPKLHVLFCLCGCLAVVYPFDWQYINPVAHMKSSAWVNKIQVLMAAASFGQTKIPRGNGPYSVGCTDLMFDHTNKGTFLRLYYPSQDNDRLDTLWIPNKEYFWGLSKFLGTHWLMGNILRLLFGSMTTPANWNSPLRPGEKYPLVVFSHGLGAFRTLYSAIGIDLASHGFIVAAVEHRDRSASATYYFKDQSAAEIGDKSWLYLRTLKQEEETHIRNEQVRQRAKECSQALSLILDIDHGKPVKNALDLKFDMEQLKDSIDREKIAVIGHSFGGATVIQTLSEDQRFRCGIALDAWMFPLGDEVYSRIPQPLFFINSEYFQYPANIIKMKKCYSPDKERKMITIRGSVHQNFADFTFATGKIIGHMLKLKGDIDSNAAIDLSNKASLAFLQKHLGLHKDFDQWDCLIEGDDENLIPGTNINTTNQHIMLQNSSGIEKYN

SEQ ID NO: 3 - DNA, 3323 bp, Homo sapien
  misc_feature 111, 178, 258, 397, 400, 505, 529, 627, 751, 949, 969,991, 1026, 1033, 1061, 1085, 1212, 1349, 1369, 1591, 1619, 1912,3056, 3153, 3154, 3263, 3275, 3281, 3302: n = A,T,C or G
gtaagtattagtgacttatttcattatgtgaaacaaacttgaagcttgggtaaatatcaatcgatatcatttggtaactattaaagaattgctgaattggttgtttagacntttcaataaggagagaattagataatctcagtttctaagtacatttagtcttactctttttaaaatngggaatgttaacgtatatagtatatatactggttatattagtctgttcttgcattgctatatggaaatacctgagactgnggtaatttataaagaaaagaggtttcattggctcacagttctgcagcctgtacaggaagcatgatgctggcatctgctcggcttctggggaggcctcaggaaacttacaattatggtggaaggcaaccggggcatgagncancttcacatggccagagccggaggaagagagggatgggtgaaggtactacacacttttaaataaccaggtctcacaagaattcactgtcacaatgacagcaccaangggggatggtgtgaaaccatgagnaaactgctcccatgatccaatcacctcccaccagtccctgcctctgacactggggattccaatttgacatgtgatttggtggggacacagatccaaanctatatcactggtaattaaaattagcattatactacatgctacttcaatctaaacaccagaatatgcctacagatttttgggggtagagctagggggaagtattccatcattaggctggggtanggaactctttaaagaaaaagtcagattatcgactgagacctgcaatatactaacctgttgagaaagaatatgagtttataaattccccaaagctataatggggtaccatgacgtgttggcaattcttgtatcctggaggtgaaaaagaactcctgataaggttttgaactctcgatgagatattacaaagcaaagatnggacctgaactcaccccttntcctatctgaaatctggtttantttaggagaagagaagaggcaggaaggaaatatantggagtnataggcaataaacactatgggctcagangaatagaggggtctcttccaacangaaaaggattcatagatgacactaaaattggattttgtaagatcaagagagtttagatagtaaggaagggtttaggtatttcaggcaaggacttcatttagcttatgtgtatgtgcaactctttctnaattctaaaacggaaataatacagaacacctaaccttccctcaaccctatttccttactaagctctcacctttgcttttgtcccttctctgctagaaaccttcaaagaatagccaatattagtcattgcatgtgctntacttctattcactctttgnactcttgtggtctagcttcctgggcccccacgggcatgccaaaacaaagacactaaaggcctgctgcctgccacaccagcagcctcttcagagtctcaaccttgctctctgcatctgttcaacattgctgaccacccctcttcctgctcccttggctactatgacccacctctttgctgcttctcctgtcatttataccactccttccatttcttctcctcntggcagttctctacagtcagatcatccnaagcttctgctcgcaattctcttgtattcctgctgtttatcttctttatgtttctgacattcaagtttcctactacaatgtggggaagagtgagagggagttaagtggccctgttttgagcttagatgactaaaagaatgatgggttgcttataagaaccagcagagtcaagaggcagagcagctttgggcaggactgggaagatgtcttttaacttcacaatttcttagcttgagaactgaggaatctgtacacagatgtctggccacacattggatgattacataatttancatgataacacagatgaaacatttgaagtagaagagattgcaaagtgagaaaagagttggtctgccagtagaatcctaaggaatgcctacattcagccaatgggaaatgggtgaagagccagtgaagaacatagagtggttagcgtcagaagggaaggtgcaatgttaaggaaacgtaaagaaaaaaagtgcaagaaggccatcaagtgtcaaatgtcccagtggtggtagaaaacgaggacttaaaaaaggccattctatttgacgttaggctattgatgaatttacagagcagtgcaaatgatatttataaagtgcttacagtttaatactgggaaaagtggtgtttgaaatctgtttcctctaaggcttaaatctaaagtgatttaaatttaaagtgactagcatcaaatacataccacgttcagtggtgagggcaggtagcaggctctggctctgagttcagggacccttcaatacagaacacattccagtattcaaactggaagtattccaattcactaaaaagcaagaatcatttcttctaaaatcaagataccaagcaagaacaagattctttgagttgtatttctagagggaagaagaatatactctgggatccctaaacaaacagcctgtgacccttgaaacacatctaagtagatcaaattacaagttttatttcttctttggttttcagtaaacagaccaacaagaccagtacctttcttacactctaactaaaaaaataataattttatcaaacaatgtgacttttaaatgtcttgttctcttttaggggttcagtccaccagaattttgctgacttcacttttgcaactggcaaaataattggacacatgctcaaattaaagggagacatagattcaaatgyagctattgatcttagcaacaaagcttcattagcattcttacaaaagcatttaggtaagaaactatttttttcatgacctaaaccagatgaatctcaggacaaagctgtctatcttaatacagctttagtactatttaaactatttccagttggtttacaatggaacaaagcagtatatcaatttgaaaacagaaatttgagaaagtcaattttgctgctttacatcctctatatcatagaaagcaaatnccaactgttaaaggtaatattctttgtatgaagcctagagtggacttccatgttgaggatactgacagcaggttgcctcactcctatcccgtttgcnnattcagctgctaaagcagccatgaggcagctgatacagagcacatcgtctctaccatcctaacggaacttgtgtaatttgtaaatctttattgccacctaggggcaccnaaactgtttaantgctcntcaaaagtttaatatgttganttaacactttatattttatag

SEQ ID NO: 4 - DNA, 20 bp, Artificial Sequence
  PCR primer
tccttacactctaactaaaa

SEQ ID NO: 5 - DNA, 20 bp, Artificial Sequence
  PCR primer
taaaccaactggaaatagtt

SEQ ID NO: 6 - DNA, 19 bp, Artificial Sequence
  Sequencing primer
ggagacatagattcaaatg